(19) United States
(12) Patent Application Publication
(10) Pub. No.: US 2024/0173451 A1
(43) Pub. Date: May 30, 2024

(54) FRAGRANCE COMPOSITIONS, METHODS OF MANUFACTURE THEREOF AND ARTICLES COMPRISING THE SAME

(71) Applicant: Exuud, Inc., Atlanta, GA (US)

(72) Inventors: Abhishek Breja, Atlanta, GA (US); Jesse David Killion, Alpharetta, GA (US)

(21) Appl. No.: 18/281,215

(22) PCT Filed: Mar. 8, 2022
(86) PCT No.: PCT/US2022/019289

§ 371 Date: Sep. 8, 2023

Related U.S. Application Data
(60) Provisional application No. 63/159,209, filed on Mar. 10, 2021.

(51) Int. Cl. A61L 9/04; A61L 9/12; A61M 21/00; C11B 9/00

(52) U.S. Cl. CPC A61L 9/048; A61L 9/042; A61L 9/044; A61L 9/122; A61L 9/125; A61M 21/00; C11B 9/0015; C11B 9/0019; C11B 9/0034; A61L 2209/134; A61M 2021/0016

(57) ABSTRACT

Disclosed herein is a fragrance composition comprising an organic aerogel; and a non-edible active fragrance compound; where the organic aerogel comprises a biopolymer or a synthetically derived organic polymer; where the organic aerogel does not contain any inorganic aerogels and wherein the organic aerogels are not blended with or dispersed in hydrogels, inorganic aerogels or with non-aerogel polymers.

### BACKGROUND

Disclosed herein are fragrance compositions, methods of manufacture thereof and articles comprising the same.

Pleasant aromas are a delightful component of the human experience, and even aromas not perceived as pleasant can often reliably trigger memories of past situations and experiences.

The ever-varying aroma patterns of a walk through the woods, a visit to the beach, flowers along one's pathway or a climb to the top of a tropical volcano are difficult to recreate but offer mood elevating potential and possible social interaction benefits to the extent such re-creation can be made successfully.

Throughout history, fragrance has been a recognized component of the best life has to offer and indeed a whole industry has arisen around so-called aromachology and aromatherapy in recent years in recognition of the numerous potential benefits of olfactory stimulation. For example, the U.S Department of Health and Human Services even uses taxpayer funding to feature an Aromatherapy page on the World Wide Web (Internet).

The challenges associated with reliable fragrance delivery, however, are as old as its usage, and continue to this day. Among others these include: fragrance being often overly intense when first applied to skin as perfume; fading of fragrance intensity while being worn as perfume; changes in compound fragrance profile with time because of variations in volatility of fragrance compounds; olfactory fatigue causing reduced sensitivity to odors, and olfactory boredom from unrealistic monotony of unchanging aroma profiles.

A host of products are currently offered for whole room fragrance delivery, these include ultrasonic, evaporation and atomizing (or nebulizing) essential oil diffusers, as well as so-called “air freshener” devices for masking unpleasant odors. These also largely suffer from unchanging, potentially fatiguing odor output and general inability to offer controllable, over time, replacement and targeted variation (or mixture) of complex fragrances with also-controllable intensity of aroma delivery over time.

Also available in relative abundance are vapor storage and delivery devices utilizing microfluidic pumps coupled to fluid reservoirs. These include devices such as “e-Cigs” that typically utilize electronically controlled heating to boil or vaporize fragrance compounds on demand. The intensity of heat applied often results in denaturing of the fragrance compounds and possibly chemical breakdown. The controllers and processing systems make these “high technology” approaches to fragrance delivery complex and thus costly. Storing fluid compounds in a fluid reservoir also creates limitations on product handling and shipping as well as the need for fluidic seals that are prone to failure during use. The need thus exists for low-cost fragrance delivery systems that overcome some of the aforementioned challenges.

### SUMMARY

Disclosed herein is a fragrance composition comprising an organic aerogel; and a non-edible active fragrance compound; where the organic aerogel comprises a biopolymer or a synthetically derived organic polymer; where the organic aerogel does not contain any inorganic aerogels and wherein the organic aerogels are not blended with or dispersed in hydrogels, inorganic aerogels or with non-aerogel polymers.

Disclosed herein too is a method of manufacturing a fragrance composition comprising blending together an organic aerogel and a non-edible active fragrance compound to form the fragrance composition; where the organic aerogel comprises biopolymer or a synthetically derived organic polymer; where the organic aerogel does not contain any inorganic aerogels and wherein the organic aerogels are not blended with or dispersed in hydrogels, inorganic aerogels or with non-aerogel polymers.

Disclosed herein too is a fragrance emitting device comprising a first container comprising a porous wall that is operative to contain an active fragrance compound that can be discharged to a surrounding atmosphere; and a mobile chamber that comprises the active fragrance compound; where the mobile chamber can be moved from a position where it is not exposed to an ambient atmosphere to another position where it is exposed to ambient atmosphere and discharges the active fragrance compound to the ambient atmosphere.

### BRIEF DESCRIPTION OF THE FIGURES

FIG. 1A is an exemplary schematic depiction of a fragrance emitting device;

FIG. 1B is another exemplary schematic depiction of the fragrance emitting device of the FIG. 1A;

FIG. 2 is a depiction of another exemplary embodiment of the fragrance emitting device;

FIG. 3 is a depiction of another exemplary embodiment of the fragrance emitting device;

FIG. 4 is a depiction of another exemplary embodiment of the fragrance emitting device;

FIG. 5 is a graph that depicts the weight loss (an evaporation rate) of the active fragrance compound versus time;

FIG. 6 is a depiction of another exemplary embodiment of the fragrance emitting device.

### DETAILED DESCRIPTION

As used herein, the term “fragrance composition” refers to at least one fragrance compound such as an active fragrance compound (for odor purposes only). The fragrance composition can be a single fragrance compound or a mixture of multiple fragrance compounds. The fragrance composition may also include other additives, carriers, diluents, solvents, surfactants, and the like.

As used herein, the term “flavorant” or “flavorant composition” refers to an aroma composition used for taste purposes. A non-edible flavorant or flavorant composition refers to an aroma composition that is not consumed for digestive purposes but is experienced primarily for taste or flavor purposes, for instance as a flavored smoke or vapor.

Disclosed herein is a fragrance composition that comprises organic aerogels and a non-edible active fragrance compound. The non-edible fragrance imparts an odor (also sometimes referred to as a scent or aroma) to other materials (e.g., solids, gases, liquids, or combinations thereof) that it contacts but (the non-edible fragrance) cannot be consumed for digestive purposes by living beings. In other words, the fragrance compound is not used as a flavorant nor is it an edible flavorant. In an embodiment, the fragrance compound is a non-edible flavorant. The fragrance composition is a non-edible composition—it is not suitable for digestive purposes by living beings.

Disclosed herein too is a fragrance emitting device that emits the active fragrance compound from the fragrance composition to the atmosphere such that a user perceives that the surrounding atmosphere contains a controlled level of the active fragrance compound. In an embodiment, the fragrance emitting device emits the active fragrance compound from the fragrance composition to the atmosphere such that a user perceives that the surrounding atmosphere contains a constant level of the active fragrance compound. In another embodiment, the fragrance emitting device emits the active fragrance compound from the fragrance composition to the atmosphere such that the amount of fragrance emitted is controlled by varying exposure to air and heat to create a variable or constant user perception. Details of the fragrance emitting device are provided later in this disclosure.

The organic aerogel comprises a biopolymer or a synthetically derived organic polymer (hereinafter synthetic polymer). The organic aerogel does not contain any inorganic aerogels such as, for example, silicate aerogels, titanate aerogels, aluminate aerogels, zirconate aerogels, or the like. The organic aerogels are not blended with or dispersed in hydrogels, inorganic aerogels, or with non-aerogel polymers in the fragrance composition. In other words, the organic aerogel is not dispersed in a matrix of hydrogels, inorganic aerogels, or non-aerogel polymers.

Biopolymers are polymers composed primarily of a few types of repeating units containing carbon which are used in or originate from living organisms. Biopolymers are natural polymers produced by the cells of living organisms. Biopolymers consist of monomeric units that are covalently bonded to form larger polymeric molecules. There are three main classes of biopolymers, classified according to the monomers used and the structure of the biopolymer formed: polynucleotides, polypeptides, and polysaccharides. The biopolymers used in the aerogels are preferably food grade aerogels (i.e., they can be digested by living beings).

Examples of biopolymers that may be used in the aerogels include cellulose, reduced crystallinity cellulose, polysaccharides, chitosan, oligochitosan, gelatin, collagen, hydroxyalkyl celluloses such as hydroxypropyl cellulose, hydroxymethyl cellulose and hydroxyethyl cellulose; sodium carboxymethyl cellulose, cellulose acetate, cellulose diacetate, cellulose triacetate, cellulose propionate, cellulose acetate butyrate, and cellulose ethers like ethyl cellulose, sugars (glucose, sucrose, lactose, galactose, fructose, mannitol, sorbitol, or a combination thereof), proteins, starch, pectin, alginate, starch sodium octenyl succinate, locust bean gum, carrageenan, agar, xanthan gum, guar gum, casein, whey protein isolate, soy protein isolate, pea protein isolate, potato protein isolate, zein, lecithin, stearic acid, beeswax, cottonseed wax, carnauba wax, milk fat, palm and palm kernel oil, or the like, or a combination thereof.

Synthetic polymers can also be used in the aerogels. The synthetic polymers are organic polymers and may be selected from a wide variety of thermoplastic polymers, blends of thermoplastic polymers, thermosetting polymers, or blends of thermoplastic polymers with thermosetting polymers. The organic polymer may also be a blend of polymers, copolymers, terpolymers, or combinations comprising at least one of the foregoing organic polymers. The organic polymer can also be an oligomer, a homopolymer, a copolymer, a block copolymer, an alternating block copolymer, a random polymer, a random copolymer, a random block copolymer, a graft copolymer, a star block copolymer, a dendrimer, a polyelectrolyte (polymers that have some repeat groups that contain electrolytes), a polyampholyte (a polyelectrolyte having both cationic and anionic repeat groups), an ionomer, or the like, or a combination thereof. The organic polymers have number average molecular weights greater than 10,000 grams per mole, preferably greater than 20,000 g/mole and more preferably greater than 50,000 g/mole.

Examples of thermoplastic polymers that can be used in the polymeric material include polyacetals, polyacrylics, polycarbonates, polyalkyds, polystyrenes, polyolefins, polyesters, polyamides, polyaramides, polyamideimides, polyarylates, polyurethanes, epoxies, phenolics, silicones, polyarylsulfones, polyethersulfones, polyphenylene sulfides, polysulfones, polyimides, polyetherimides, polytetrafluoroethylenes, polyetherketones, polyether ether ketones, polyether ketone ketones, polybenzoxazoles, polyoxadiazoles, polybenzothiazinophenothiazines, polybenzothiazoles, polypyrazinoquinoxalines, polypyromellitimides, polyguinoxalines, polybenzimidazoles, polyoxindoles, polyoxoisoindolines, polydioxoisoindolines, polytriazines, polypyridazines, polypiperazines, polypyridines, polypiperidines, polytriazoles, polypyrazoles, polycarboranes, polyoxabicyclononanes, polydibenzofurans, polyphthalides, polyacetals, polyanhydrides, polyvinyl ethers, polyvinyl thioethers, polyvinyl alcohols, polyvinyl ketones, polyvinyl halides, polyvinyl nitriles, polyvinyl esters, polysulfonates, polysulfides, polythioesters, polysulfones, polysulfonamides, polyureas, polyphosphazenes, polysilazanes, polypropylenes, polyethylenes, polyethylene terephthalates, polyvinylidene fluorides, polysiloxanes, or the like, or a combination thereof.

Examples of thermosetting polymers suitable for use as hosts in emissive layer include epoxy polymers, unsaturated polyester polymers, polyimide polymers, bismaleimide polymers, bismaleimide triazine polymers, cyanate ester polymers, vinyl polymers, benzoxazine polymers, benzocyclobutene polymers, acrylics, alkyds, phenol-formaldehyde polymers, novolacs, resoles, melamine-formaldehyde polymers, urea-formaldehyde polymers, hydroxymethylfurans, isocyanates, diallyl phthalate, triallyl cyanurate, triallyl isocyanurate, unsaturated polyesterimides, resorcinol formaldehyde, phenol formaldehyde, melamine formaldehyde, cresol formaldehyde, phenol furfuryl alcohol, or the like, or a combination thereof. The biopolymer aerogels are preferred. Alginate based aerogels are preferred amongst the biopolymers.

The aerogels may be manufactured using supercritical extraction, freeze drying or a combination thereof. Details of the manufacturing of alginate aerogels will not be provided here but can be accessed elsewhere.

Suitable organic aerogel particles have a volume-average average pore diameter of preferably 20 nanometers to 4 microns, preferably 50 nm to 1 micrometer.

Suitable organic aerogel particles preferably have a porosity of 75 to 99% by volume, more preferably 87% to 97% by volume. The porosity by volume is the specific proportion of the total volume of the aerogel particle comprising pores.

Due to their small pore size and high porosity, aerogel particles exposed to liquids that wet the aerogel material are subject to extreme forces due to surface tension that can break down the structure of the aerogel. Typical inorganic aerogels such silica-based aerogels break in brittle failure due to surface tension forces if exposed to a liquid that wets the aerogel. Thus, loading and unloading silica aerogels with fragrance compound may result in the disintegration of inorganic aerogel particles into multiple smaller particles that can be hard to contain and would present an inhalation risk to humans and other animals. On the contrary, suitable organic aerogel particles do not exhibit brittle failure even in the presence of extreme surface tension forces that break down the structure of the aerogel. Instead, organic aerogel particles simply shrink (particle volume reduces) without breaking into multiple particles and can become materially harder and more robust as the aerogel matrix is restructured during shrinkage. This novel behavior of organic aerogels is beneficial in the present application for sorting used particles from new particles, visually verifying the evaporation rate by examining particle volume, and eliminating inhalation or other health risks associated with inorganic, brittle aerogel materials.

Organic aerogel particles can shrink in volume both during loading with fragrance compound and as the fragrance compound evaporates. During loading of the fragrance compound into the fragrance composition, the organic aerogel particles preferably shrink by 0% to 25% by volume, more preferably by 1% to 10% by volume, and more preferably by 2% to 5% by volume. During evaporation of the fragrance compound from the fragrance composition, the organic aerogel particles preferably shrink by 40% to 95% by volume, more preferably by 60% to 92% by volume, and more preferably by 80% to 90% by volume.

The organic aerogel particles have an average particle size of 100 micrometers to 6 millimeters, preferably 500 micrometers to 5 millimeters, more preferably, and more preferably 1 millimeter to 4 millimeters.

Due to their small pore size and high porosity, aerogel particles loaded with fragrance compound can retain a very high proportion of liquid compound by volume without requiring any particular provision to prevent leakage. Thus, the fragrance composition once created can be handled, transported, and stored as a matrix of solid particles. This novel characteristic of the fragrance composition leads to significant advantages and cost savings for handling, manufacturing, distribution, packaging, storing, and utilizing fragrance compounds.

The aerogel particles are present in the fragrance composition in an amount of 1 to 20% by volume, preferably 2 to 12% by volume, and more preferably 3 to 8% by volume, based on the total volume of the fragrance composition. The aerogel particles are present in the fragrance composition in an amount of 1 to 40 wt %, preferably 5 to 25 wt %, and more preferably 8 to 17 wt %, based on a total weight of the fragrance composition.

As noted above, the fragrance composition comprises aerogels and an active fragrance compound. The active fragrance compound is also referred to as a scent, an odor, a perfume, a non-edible flavor, or a fragrance. The active fragrance compound is preferably an aromatic compound in a solvent. The solvent is preferably environmentally friendly and safe for inhalation and/or ingestion.

The intensity and longevity of a fragrance composition is based on the concentration, intensity, and longevity of the aromatic compounds or oils used. As the percentage of aromatic compound in the fragrance composition increases so does the intensity and longevity of the fragrance composition.

The active fragrance compound preferably contains a blend of so-called fragrance notes that vary in immediacy of impression to the nose. So-called top notes are those fragrance compounds which provide the most immediate impression and are typically characterized by the relatively highest volatility. So-called middle or heart notes are those that are typically perceived after the immediacy of the top notes and are characterized by somewhat lower volatility. So-called base notes tend to be compounds that have even lower volatility and thus create a more subtle but enduring impression. These notes are carefully balanced with knowledge of the evaporation characteristics and interplay of the fragrance compounds and solvents/diluents. A note is another manner of describing the odor (the fragrance) that the inhaler perceives upon inhalation of the atmosphere into which the active fragrance compound is released.

The top notes are also called the head notes. These are fragrances that are perceived immediately on the application of the fragrance compound. Top notes consist of small, light molecules that evaporate quickly. They form a person's initial impression of a fragrance and thus are very important in the selling of a perfume. Examples of top notes include mint, lavender and coriander.

The middle notes are also referred to as heart notes. The middle notes of a perfume are typically not as immediately perceived as the top note. The middle note compounds form the “heart” or main body of a perfume and act to mask the often unpleasant initial impression of base notes, which become more pleasant with time. Examples of middle notes include seawater, sandalwood and jasmine.

The fragrance composition also comprises a fragrance that has base notes. The base notes represent the scent of a perfume that endures the longest but are often less immediate than the top and middle notes. The base and middle notes together are the main theme of a perfume. Base notes bring depth and solidity to a perfume. Compounds of this class of scents are typically rich and “deep” and are sometimes not perceived for several minutes after application. Examples of base notes include tobacco, amber and musk.

The scents in the top and middle notes are influenced by the base notes; conversely, the scents of the base notes will be altered by the types of fragrance materials used as middle notes. Manufacturers who publish perfume notes typically do so with the fragrance components presented as a fragrance pyramid. Fragrance compounds are thus developed to create certain complex fragrance profiles or characters that are pleasant to the inhaler. Due to the difference in volatility of top, middle, and base notes, the release of the components of the fragrance compound into the air is not consistent over time and the perception of a fragrance profile often changes for a long time after initial application. Although generally accepted by fragrance users due to its ubiquity, the change in character or profile with time is in general undesirable and requires significant tradeoffs be made by fragrance compounders to overcome this limitation. For instance, certain diluents, fixatives, and evaporation mediating compounds are frequently employed to try to maintain more consistent fragrance profile perception over time.

As noted above, the active fragrance compound may be released into the ambient atmosphere (the surroundings) in such a way that a person inhaling the atmosphere perceives that there is a fairly constant fragrance profile (with the passage of time) in the atmosphere. To counteract olfactory adaptation and the naturally uneven evaporation rate of top, middle, and base notes, this may mean that the concentration of the active fragrance compound is varied (i.e., increased and/or decreased) over time to create the olfactory perception of a fairly constant fragrance profile in the atmosphere with the passage of time.

In another embodiment, the olfactory perception of a constant fragrance profile of the active fragrance compound in the atmosphere is created by maintaining a constant concentration of the active fragrance compound in the atmosphere. This is done by emitting a uniform amount of the active fragrance compound at different intervals over a period of time.

In yet another embodiment, a first active fragrance compound is released to the atmosphere from a first fragrance composition in a first interval of time, while a second active fragrance compound is released to the atmosphere from a second fragrance composition in a second interval of time. The first active fragrance compound may be the same or different from the second active fragrance compound. The first fragrance composition may be the same or different from the second fragrance composition. The first interval of time may be the same or different from the second interval of time.

Various sources of fragrance are described below.

Plants have long been used in perfumery as a source of essential oils and aroma compounds. These aromatics are usually secondary metabolites produced by plants as protection against herbivores, infections, as well as to attract pollinators. The sources of these compounds may be derived from various parts of a plant. Some of these are detailed below.

Bark: Commonly used barks include cinnamon and cascarilla. The fragrant oil in sassafras root bark is also used either directly or purified for its main constituent, safrole, which is used in the synthesis of other fragrant compounds.

Flowers: Flowers and blossoms are one of the most common sources of perfume aromatics. This includes the flowers of several species of rose and jasmine, as well as osmanthus, plumeria, mimosa, tuberose, narcissus, scented geranium, cassie, ambrette as well as the blossoms of citrus and ylang-ylang trees.

Fruits: Fresh fruits such as apples, strawberries, cherries rarely yield the expected odors when extracted; if such fragrance notes are found in a perfume, they are more likely to be of synthetic origin. Notable exceptions include blackcurrant leaf, litsea cubeba, vanilla, and juniper berry. The most commonly used fruits yield their aromatics from the rind; they include citrus such as oranges, lemons and limes. Although grapefruit rind is still used for aromatics, more and more commercially used grapefruit aromatics are artificially synthesized since the natural aromatic contains sulfur and its degradation product is quite unpleasant in smell.

Leaves and twigs: Commonly used for perfumery are lavender leaf, patchouli, sage, violets, rosemary, and citrus leaves. Sometimes leaves are valued for the “green” smell they bring to perfumes, examples of this include hay and tomato leaf.

Resins: Resins are widely used in incense and perfumery. Highly fragrant and antiseptic resins and resin-containing perfumes are used by many cultures as medicines for a large variety of ailments. Commonly used resins in perfumery include labdanum, frankincense/olibanum, myrrh, balsam of Peru, benzoin. Pine and fir resins are a particularly valued source of terpenes used in the organic synthesis of many other synthetic or naturally occurring aromatic compounds. Some of what is called amber and copal in perfumery today is the resinous secretion of fossil conifers.

Roots, rhizomes and bulbs: Commonly used terrestrial portions in perfumery include iris rhizomes, vetiver roots, various rhizomes of the ginger family.

Seeds: Commonly used seeds include tonka bean, carrot seed, coriander, caraway, cocoa, nutmeg, mace, cardamom, and anise.

Woods: Woods are often used to provide the base notes to a perfume, wood oils and distillates are indispensable in perfumery. Commonly used woods include sandalwood, rosewood, agarwood, birch, cedar, juniper, and pine. These are used in the form of macerations or dry-distilled (rectified) forms.

Animal fragrances may also be used singly or in conjunction with other fragrances in the fragrance composition.

Musk pods derived from male musk deer may be used in the fragrance composition.

Ambergris: Lumps of oxidized fatty compounds, whose precursors were secreted and expelled by sperm whales. Ambergris should not be confused with yellow amber, which is used in jewelry. Because the harvesting of ambergris involves no harm to its animal source, it remains one of the few animalic fragrancing agents around which little controversy now exists.

Castoreum: Obtained from the odorous sacs of the North American beaver.

Civet: Also called civet musk, this is obtained from the odorous sacs of the civets, animals in the family Viverridae, related to the mongoose.

Hyraceum: Commonly known as “Africa stone”, is the petrified excrement of the rock hyrax.

Honeycomb: From the honeycomb of the honeybee. Both beeswax and honey can be solvent extracted to produce an absolute. Beeswax is extracted with ethanol and the ethanol evaporated to produce beeswax absolute.

Musk: Originally derived from a gland (sac or pod) located between the genitals and the umbilicus of the Himalayan male musk deer Moschus moschiferus, it has now mainly been replaced by the use of synthetic musks sometimes known as “white musk”. Other natural sources Lichens: Commonly used lichens include oakmoss and treemoss thalli.

Seaweed: Distillates are sometimes used as essential oil in perfumes. An example of a commonly used seaweed is Fucus vesiculosus, which is commonly referred to as bladder wrack.

### Synthetic Sources

Many modern perfumes contain synthesized odorants. Synthetics can provide fragrances which are not found in nature. For instance, calone, a compound of synthetic origin, imparts a fresh ozonous metallic marine scent that is widely used in contemporary perfumes. Synthetic aromatics are often used as an alternate source of compounds that are not easily obtained from natural sources. For example, linalool and coumarin are both naturally occurring compounds that can be inexpensively synthesized from terpenes. Orchid scents (typically salicylates) are usually not obtained directly from the plant itself but are instead synthetically created to match the fragrant compounds found in various orchids.

One of the most commonly used classes of synthetic aromatics by far are the white musks. These materials are found in all forms of commercial perfumes as a neutral background to the middle notes.

The active fragrance compound may be selected from lactic acid, 2-hydroxydecanoic acid, 2-hydroxyoctanoic acid and glycolic acid, beta-hydroxy acids such as beta-hydroxy salicylic acid, avobenzone, benzoate-4-methylbenzylidene, cinoxate, dioxybenzone, homosalate, menthyl anthranilate, mexoryl SX, drometrizole trisiloxane, octocrylene, octyl methoxycinnamate, ethylhexyl salicylate, oxybenzone, padimate O, p-aminobenzoic acid (PABA), phenylbenzimidazole sulfonic acid, sulisobenzone, titanium trolamine salicylate, salicylic acid, a retinoic acid, benzoyl peroxide, hydroquinone, arbutun, a plant extract containing arbuten, kojic acid, azelaic acid, glycyrrhetic acid, levulinic acid, 2-cyano-3,3-diphenylacrylic acid, sodium benzotriazolyl butylphenol sulfonate, ethyl-2-cyan-3,3-diphenylacrylate, 2-t-butyl-6-(5-chloro-2H-benzotriazol-2-yl)-p-cresol, 2-(2-H-benzotriazol-2-yl)-4-methylphenol, benzophenone-12, bornelone, 2-benzotriazolyl-4-tert-octylphenol, dihydroxyacetone, erythrulose, dihydroxyacetone-ortho-ethyl acetate, canthaxanthin, afamelanotide, minoxidil, finasteride, dutasteride, ketoconazole, zinc pyrithione, selenium sulfide, clotrimazole, tea tree oil, piroctone olamine, an amphetamine, an antihistamine, methylphenidate, oxymetazoline, tetrahydrolzoline hydrochloride, psilocybin calcium, thioglycolate, sodium thioglycolate, thioglycolic acid, ammonium thioglycolate, butyl thioglycolate, ethanolamine thioglycolate, glyceryl thioglycolate, isooctyl thioglycolate, isopropyl thioglycolate, magnesium thioglycolate, methyl thioglycolate, potassium thioglycolate, aluminum zirconium tetrachlorohydrex gly, aluminum chlorohydrate, aluminum chloride, resorcinol, 1-napthol, p-aminophenol, p-phenylenediamine, salts of p-phenylenediamine, 4-amino-2-hydroxytoluene, phenoxyethanol, N,N-diethyl-m-toluamide, p-menthane-3,8-diol, nepetalactone, citronella oil, permethrin, neem oil, bog myrtle extract, or a combination thereof.

In an embodiment, the active fragrance compound may be a fragrance oil such as, for example, cottonseed oil, lavender essential oil, vanilla oil, cinnamon oil, citronella oil, goji berry fragrance oil, orange oil, mandarin orange oil, apple seed oil, Caribbean teakwood oil, cedar oil, sandalwood oil, juniper oil, nutmeg oil, star anise oil, patchouli oil, rose oil, clove oil, saffron oil, lavender oil, rosemary oil, clary sage oil, lemon oil, peppermint oil, sweet basil oil, bergamot oil, camellia oil (or tea seed oil), blue chamomile oil, catnip oil, bay leaf oil, clementine oil, coffee essential oil, coconut oil, oregano oil, ylang-ylang oil, neroli essential oil, bergamot essential oil, rose petal oil, jasmine essential oil, vetiver essential oil, citrus essential oil, olibanum essential oil, petitgrain oil, sweet orange oil, myrrh essential oil, coriander seed oil, frankincense oil, or a combination thereof.

The fragrance composition may contain the active fragrance compound in an amount of 60 to 99 wt %, preferably 75 to 95 wt %, and more preferably 83 to 91 wt %, based on the total weight of the fragrance composition.

In an embodiment, the fragrance composition may comprise an optional solvent. Solvents, if used, facilitate the uniform blending of the fragrance compounds, diffusion and ingression of the active fragrance compound into the pores of the organic aerogel for long term storage, as well as acting as diluents to emitted fragrance intensity, and augmenting the evaporation and diffusion rates from and within the aerogel. Suitable solvents are aqueous solvents that are biocompatible, i.e., solvents that are non-toxic to and ingestible by living beings and preferably compatible with water. Examples of suitable solvents are water, ethanol, glycerol, isopropylidene glycerol, polar glycerol derivatives such as, for example, glycerol, 1,2- and 1,3-propanediol, or combinations thereof.

The solvent is preferably used in amounts of 5 to 80 wt %, preferably 7 to 75 wt %, and more preferably 9 to 70 wt %, based on the total weight of the fragrance composition. In an embodiment, when the fragrance composition contains both an active fragrance compound and a solvent, the weight ratio of the active fragrance compound to the solvent varies from 6:1 to 1:6.

In one embodiment, the fragrance composition may comprise an optional surfactant. The optional surfactant may also be used to compatibilize the active fragrance compound with the organic aerogel and facilitate diffusion and ingression of the active fragrance compound into the pores of the aerogel for long term storage. The surfactant is generally environmentally safe, non-toxic to living beings and compatible with the solvent and the active fragrance compound. The surfactant, if used, is present in the fragrance composition in an amount of 0.5 to 5 wt %, preferably 1 to 4 wt %, preferably 2 to 3 wt %, based on the total weight of the fragrance composition.

The fragrance composition is manufactured by blending the organic aerogel particles together with the active fragrance compound, any desirable solvent, and surfactants in a reactor. Capillary action causes diffusion and ingression of the active fragrance compound into the organic aerogel. The diffusion and ingression of the active fragrance compound into the organic aerogel may occur from room temperature to a temperature below the boiling point of the solvent or the boiling point of the active fragrance compound. The pressure may vary from ambient pressure to pressures of 5000 kiloPascal. Preferable temperature for the diffusion is room temperature and preferable pressure is ambient pressure.

The fragrance composition may be stored under ambient pressure and temperature for extended periods of time. It is preferably stored in a manner to prevent leakage of the active fragrance compound from the aerogel particles.

The fragrance composition offers a wide range of property combinations that can be used advantageously in various applications such as fragrance diffusers (a fragrance emitting device), antiperspirants, deodorants, cleaners, soaps, perfumes, colognes, candles, furniture abrasives, chemical products, vaporizers, or the like.

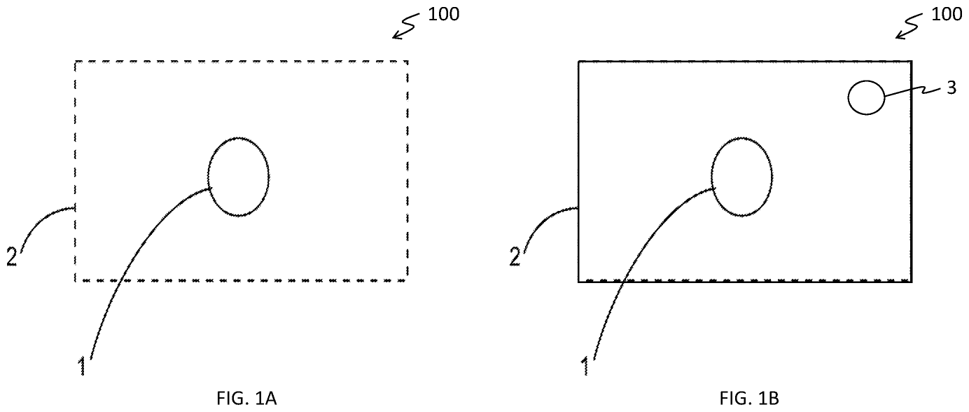

The fragrance composition may be loaded into a fragrance emitting device that can be used to perfume the ambient environment as desired. The remainder of this document will detail one exemplary embodiment of a fragrance emitting device. FIG. 1A depicts one exemplary embodiment of a fragrance emitting device 100. The device 100 comprises a first container 2 comprising the fragrance composition 1. The fragrance composition may be present in the first container as a single particle or a plurality of particles. The first container 2 comprises porous walls (depicted by dotted lines) through which the active fragrance compound may be discharged to the surrounding ambient atmosphere, or alternatively, may have a solid non-porous wall with one or more ports 3 through which the active fragrance compound may be discharged to the atmosphere. The first container 2 may be manufactured from a material that preferably does not react with the active fragrance compound and can withstand temperatures of operation of the fragrance emitting device 100. FIG. 1B depicts a fragrance emitting device 100 where the container 2 contains the fragrance emitting composition 1. The container 2 contains solid walls and one or more ports 3 through which a user may inhale the active fragrance compound (e.g., where the active fragrance compound is a non-edible flavorant). In an embodiment, the user may put his/her lips to the port 3 and inhale the flavorant.

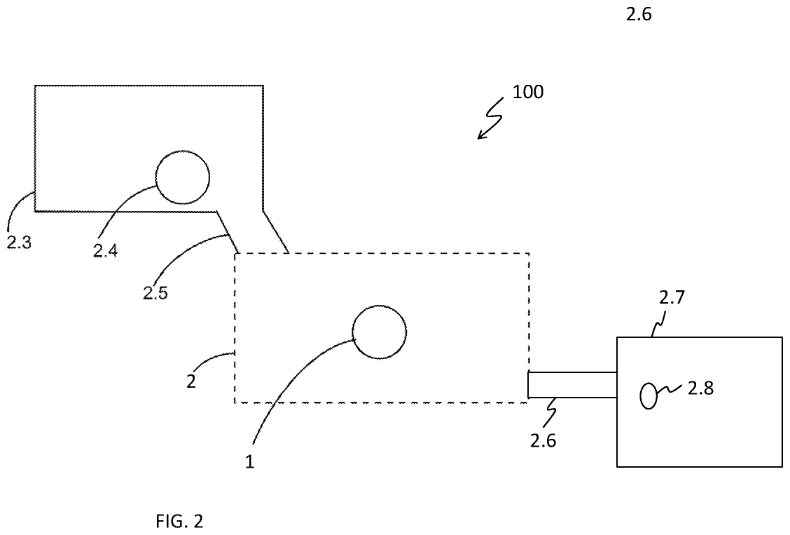

FIG. 2 depicts another exemplary embodiment of the fragrance emitting device 100 where the first container 2 from the FIGS. 1A or 1B is in operative communication with a second container 2.3. It should be noted that the first container 2 may have porous walls or solid walls with one or more ports as depicted in the FIG. 1B. The second container 2.3 comprises a second fragrance composition 2.4 and is provided with a passage 2.5 for transporting the fragrance composition from the second container 2.3 to the first container 2. The first container 2 lies downstream of the second container 2.3. The second container 2.3 serves as a storage cell for the fragrance composition until it is desired to transport it to the first container 2 for emitting fragrance. Fresh fragrance composition is then charged to the first container 2 via passage 2.5 to replace the spent fragrance composition in the first container 2.

In an embodiment, the first container 2 can be unsealed to emit the active fragrance compound to the ambient surroundings. In an embodiment, the first container 2 may be provided with an agitator to facilitate air circulation through the aerogel particles in the fragrance composition. The agitator also exposes parts of the aerogel particles to the atmosphere in the first container 2. When the fragrance composition in the first container 2 is spent (i.e., has lost its active fragrance compound) it can be optionally removed through second port 2.6 and can be replenished with reserve fragrance composition stored in the second container 2.3. In an embodiment, the second container, 2.3 may be provided with an agitator (not shown) to facilitate the transportation of the second fragrance composition through the passage 2.5 into the first container 2. Alternatively, the second fragrance composition 2.4 may be discharged from the second container 2.3 into the first container 2 via gravity (e.g., a chute) or mechanical force such as, for example, an augur (not shown).

In an embodiment, the reserve fragrance composition (a second fragrance composition) 2.4 stored in the second container 2.3 may be the same or different from the exposed fragrance composition (a first fragrance composition) 1 stored in the container 2. In an embodiment, each successive replenishment of the second container 2.3 may be the same or different from a preceding fragrance composition.

In yet another embodiment with regard to FIG. 2, the spent fragrance composition may be discharged to a reservoir 2.7 (a third container 2.7) via passage 2.6. The passage 2.6 and the reservoir 2.7 lies downstream of the first container 2 and the spent fragrance composition may be discharged to the reservoir 2.7 via gravity (e.g., a chute) or mechanical force such as, for example, an auger (not shown). In an embodiment, the passage 2.6 has a port of a certain size that only allows beads that have shrunk due to loss of fragrance to pass through. In yet another embodiment, container 2 has a progressively reducing cross-sectional area to allow progressive motion of the aerogel particles through the passage as the fragrance evaporates and the aerogel particle shrinks until it is able to pass through passage 2.6. In other words, the container 2 and passage 2.6 are designed to serve as a sieve and/or as a regulator of fragrance composition (aerogel particle) velocity and motion during the fragrance emitting process. The shrinkage of the aerogel particles may thus be used to determine when the aerogel particles are devoid of the fragrance composition. The spent fragrance composition 2.8 is stored in the reservoir 2.7. The spent fragrance composition 2.8 is discarded when desired.

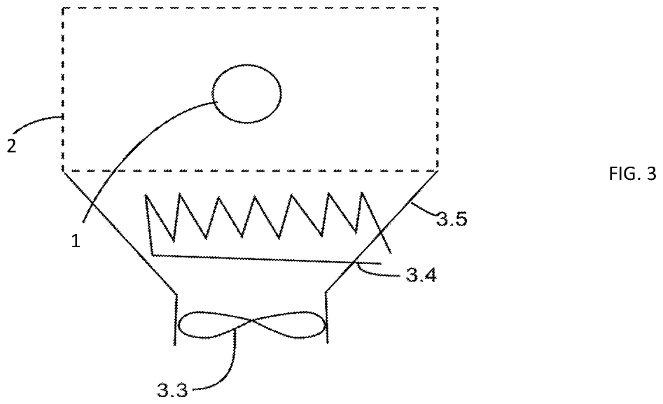

In another embodiment, with reference to the FIGS. 1A and 2, the first container may be provided with heating equipment and an aeration device to facilitate the discharge of the active fragrance compound from the aerogel particles in the fragrance composition. FIG. 3 depicts one embodiment where the where the first container 2 with the first fragrance composition 1 is provided with a heater 3.4 and/or a first source of forced air 3.3. The heater 3.4 and/or the first source of forced air 3.3 may be disposed in a supplemental chamber 3.5 that is in contact with the first container 2. The heater 3.4 may be used to heat the atmosphere that contacts the fragrance composition 1 disposed on the first container 2. The use of air with elevated temperature facilitates greater discharge of the active fragrance compound from the aerogel to the ambient atmosphere. In an alternative embodiment, the aerogel and active fragrance compound are both heated directly by the heater 3.4 instead of the air being heated and then brought into contact with the fragrance composition.

In another embodiment, the forced air flow entering the first container 2 is directed in a circulating motion to pass over the fragrance composition 1 multiple times (cyclonic flow pattern) before leaving the first container 2. The first container 2 may also be provided with a nozzle (not shown) to direct air exiting the first container 2 to promote fragrance from the fragrance emitting device to the surrounding environment. The source of forced air 3.3 can also be used to regulate the flow of air through the first container and to facilitate evaporation of the active fragrance compound to the ambient atmosphere. In summary, controlled heating, controlled air flow or a combination thereof may be used to facilitate controlled release of the active fragrance compound from the aerogels in the fragrance composition.

It is to be noted that the embodiments of the FIGS. 1A and 2 can be combined with the embodiment of the FIG. 3. In other words, the supplemental chamber 3.5 with the heater 3.4 and the source of forced air 3.3 (from the FIG. 3) may contact the first container 2 in either the FIG. 1A or the FIG. 2.

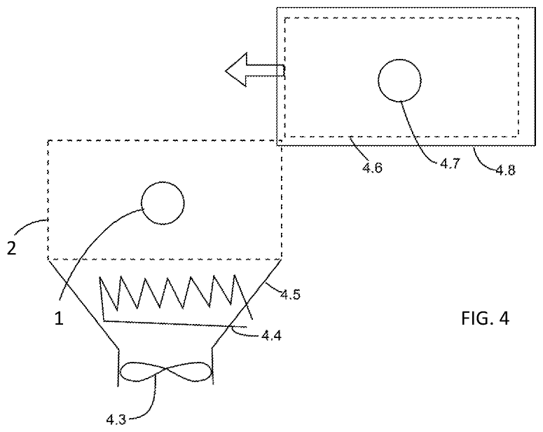

In yet another embodiment, the fragrance emitting device 100 comprises a mobile chamber 4.6 (that contains the fragrance composition 4.7) that can be moved from a position where it is not exposed to an ambient atmosphere 4.8 to another position where it is exposed to ambient atmosphere and discharges the active fragrance compound to the ambient atmosphere. FIG. 4 depicts another embodiment, where a second container 4.6 may be moved to a position such that any airflow directed to first container 2 (that contains the fragrance composition) may be now directed to the second container 4.6. Fragrance composition contained in the second container 4.6 is now exposed to airflow from supplemental chamber 4.5 and the active fragrance compound from the aerogel particles is discharged to the atmosphere. In another embodiment any and all containers (first container 2 and second container 4.6) may subsequently be moved to a position where they are no longer exposed to the atmosphere after the aerogel particle loaded with fragrance composition has discharged some of its fragrance compound to the ambient surroundings. In other words, the mobile chamber containing the fragrance composition can be moved from a position where it is exposed to an ambient atmosphere and discharges the active fragrance compound to the ambient atmosphere to another position where it is not exposed to ambient atmosphere and does not discharge the active fragrance compound to the ambient atmosphere.

While the FIG. 4 depicts a single mobile chamber 4.6 that contacts the first container 2, it can be envisioned that a plurality of mobile chambers (not shown) can be brought into contact with the first container 2 in sequential fashion. Each successive chamber can contain the same fragrance composition or a different fragrance composition from the preceding chamber. This permits different sequences of active fragrance compounds to be brought into the air flow field. In an embodiment, the time period during which each successive chamber is exposed to the air flow field (in contact with the first container 2) may be varied. It is to be noted that the fragrance emitting device of the FIG. 4 may be used to emit the active fragrance compound even when it is not absorbed into an aerogel. The fragrance emitting device can emit an active fragrance compound whose fragrance (composition) varies with time.

In another embodiment, the fragrance emitting device can be designed such that the active fragrance compound is subjected to a circumferentially directed air flow; where the circumferentially directed air flow can contact the fragrance composition a plurality of times as it (the air flow) travels round the fragrance emitting device.

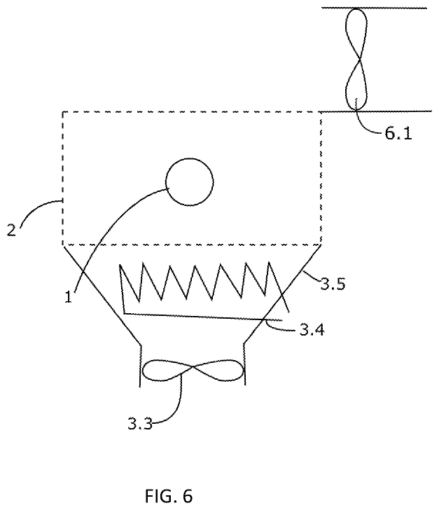

In another embodiment, depicted in FIG. 6, the fragrance emitting device can be designed with a second source of forced air 6.1 that is designed to mix a high-volume flow of ambient air with the fragrance-rich air leaving the first container 2 and/or 4.6 for the purpose of enhancing the dispersion of the fragrance into the ambient space using higher velocity and/or higher volumetric airflow than that which occurs over the fragrance composition 1. The second source of forced air 6.1 may displace the air in a different direction from the first source of forced air. It is to be noted, that in the FIGS. 2, 3, 4 and 6, the container may have porous or solid non-porous walls. The container with solid walls generally contains one or more ports through which a user may inhale the active fragrance compound, especially a non-edible flavorant.

In an embodiment, the fragrance emitting device emits the active fragrance compound from the fragrance composition to the atmosphere such that a user perceives that the surrounding atmosphere contains a constant level of the active fragrance compound with the passage of time. This means that the fragrance emitting device may discharge a constant amount of the active fragrance compound per unit time, or alternatively, may discharge a variable amount of the active fragrance compound per unit time to create the perception in the user (the inhaler) that there is a constant amount of the active fragrance compound in the atmosphere.

In an embodiment, the fragrance emitting device 100 may be a wearable device. It can be worn on the body of the wearer such as on the shirt (as a broach), an arm band, a wrist band, or carried in a pocket (like a cell phone). The fragrance emitting device disclosed herein is advantageous because it can be used to discharge a continuous and or continuously variable stream of the active fragrance compound or compounds to the atmosphere. It can be programmed via communication with a microprocessor to discharge different fragrances to the atmosphere during different parts of the day. The microprocessor can provide the fragrance emitting device with programmable aspects. For example, a user can program which fragrances would be released during different times during the day and night. The release time period can also be programmed. The microprocessor would facilitate receiving feedback from the user and adjust operation based on this feedback. The microprocessor can use algorithms that enable smart operation of the fragrance emitting device (i.e., the device is a learning device) using this feedback.

By adjusting the temperature, airflow rate, and exposure of containers to the ambient surroundings, different notes can be discharged at different rates during the day thus avoiding olfactory fatigue and keeping the ambient atmosphere feeling new and fresh.

The discharge rate of fragrance into air can be controlled by varying the flow rate and temperature of air passed over the fragrance compound. However, the flow rates and temperatures that are optimized for extracting fragrance from the fragrance compound are not, in general, optimized for dispersion into the space where the user will perceive the fragrance. Air flow that contains the evaporated fragrance, evaporation flow, can thus be combined with another variably controlled flow of air, dispersion airflow, that is optimized for spreading into the ambient by a second, independently controlled air mover. The air mover designed for dispersion airflow into the space may be a relatively high volume, low pressure-drop device that efficiently moves a large volume of air. The air mover designed for the evaporation airflow may be lower volumetric flow rate, higher pressure-drop. The evaporation airflow may also be heated. The dispersion airflow may not be heated to save energy.

### Example

This example was conducted to demonstrate the manufacture of the fragrance composition. 0.143 grams (g) of an alginate aerogel having an average particle size of 4.0 mm diameter were loaded with 0.717 g of a solution that contains an active fragrance compound comprising triplal, isocyclocitral, leaf alcohol, and amyl salicylate and solvent isopropylidene glycerol. The isopropylidene glycerol is present in an amount of approximately 0.215 g, the active fragrance compound in the amount of 0.502 g.

The active fragrance compound is mixed with the alginate aerogel particles at room temperature and atmosphere pressure for a period of approximately 15 minutes. Mixing was done in a sealed polyethylene container that remained sealed until use.

The particles with the fragrance compound were loaded into a vented chamber and placed in a steady flow of ambient air for a period of 5 hours and 30 minutes. The weight loss and evaporation rate are recorded in the table below. After 5 hours and 30 minutes, the size of the particles were measured using digital image processing of microscope images and were found to be on average 2.2 mm diameter representing an 83% reduction in particle volume and evaporation of approximately 88% of the total fragrance compound. The readings are shown in the Table below.

TABLE

| time | mass loss | evap rate | total evap | total evap |
|---|---|---|---|---|
| hr | g | g/hr | g | % |
| 0.00 | | | 0 | 0% |
| 0.25 | 0.116 | 0.46 | 0.116 | 16% |
| 0.50 | 0.079 | 0.32 | 0.195 | 27% |
| 1.00 | 0.164 | 0.33 | 0.359 | 50% |
| 1.50 | 0.106 | 0.21 | 0.465 | 65% |
| 2.00 | 0.072 | 0.14 | 0.537 | 75% |
| 3.50 | 0.075 | 0.05 | 0.612 | 85% |
| 5.50 | 0.017 | 0.01 | 0.629 | 88% |

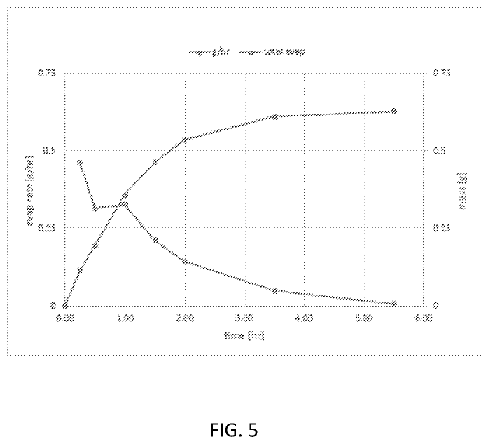

The results from the Table above are shown in graphical form in the FIG. 5. FIG. 5 is a graph of the weight loss of the active fragrance compound versus time and the evaporation rate of the active fragrance compound versus time. From the graph is may be seen that the weight loss of the active fragrance compound is greater at the start of the exposure to the ambient atmosphere than later in the process.

While the invention has been described with reference to some embodiments, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiments disclosed as the best mode contemplated for carrying out this invention, but that the invention will include all embodiments falling within the scope of the appended claims.

What is claimed is:

1. A fragrance composition comprising:
an organic aerogel; and
a non-edible active fragrance compound; where the organic aerogel comprises a biopolymer or a synthetically derived organic polymer; where the organic aerogel does not contain any inorganic aerogels and wherein the organic aerogels are not blended with or dispersed in hydrogels, inorganic aerogels or with non-aerogel polymers; where the non-edible active fragrance compound is a non-edible flavorant or a non-edible fragrance.

2. The fragrance composition of claim 1, where the organic aerogel is present in an amount of 1 to 40 wt %, based on a total weight of the fragrance composition.

3. The fragrance composition of claim 1, where the active fragrance compound is present in an amount of 60 to 99 wt %, based on a total weight of the fragrance composition.

4. The fragrance composition of claim 1, where the organic aerogel has an average particle size of 500 micrometers to 5 millimeters.

5. The fragrance composition of claim 1, where the biopolymer is derived from a living organism.

6. The fragrance composition of claim 1, where the biopolymer comprises cellulose, reduced crystallinity cellulose, polysaccharides, chitosan, oligochitosan, gelatin, collagen, hydroxypropyl cellulose, hydroxymethyl cellulose, hydroxyethyl cellulose, sodium carboxymethyl cellulose, cellulose acetate, cellulose diacetate, cellulose triacetate, cellulose propionate, cellulose acetate butyrate, ethyl cellulose, glucose, sucrose, lactose, galactose, fructose, mannitol, sorbitol, proteins, starch, pectin, alginate, sodium octenyl succinate, locust bean gum, carrageenan, agar, xanthan gum, guar gum, casein, whey protein isolate, soy protein isolate, pea protein isolate, potato protein isolate, zein, lecithin, stearic acid, beeswax, cottonseed wax, carnauba wax, milk fat, palm and palm kernel oil, or a combination thereof.

7. The fragrance composition of claim 1, where the synthetically derived organic polymer is a thermoplastic and comprises polyacetals, polyacrylics, polycarbonates, polyalkyds, polystyrenes, polyolefins, polyesters, polyamides, polyaramides, polyamideimides, polyarylates, polyurethanes, epoxies, phenolics, silicones, polyarylsulfones, polyethersulfones, polyphenylene sulfides, polysulfones, polyimides, polyetherimides, polytetrafluoroethylenes, polyetherketones, polyether ether ketones, polyether ketone ketones, polybenzoxazoles, polyoxadiazoles, polybenzothiazinophenothiazines, polybenzothiazoles, polypyrazinoquinoxalines, polypyromellitimides, polyguinoxalines, polybenzimidazoles, polyoxindoles, polyoxoisoindolines, polydioxoisoindolines, polytriazines, polypyridazines, polypiperazines, polypyridines, polypiperidines, polytriazoles, polypyrazoles, polycarboranes, polyoxabicyclononanes, polydibenzofurans, polyphthalides, polyacetals, polyanhydrides, polyvinyl ethers, polyvinyl thioethers, polyvinyl alcohols, polyvinyl ketones, polyvinyl halides, polyvinyl nitriles, polyvinyl esters, polysulfonates, polysulfides, polythioesters, polysulfones, polysulfonamides, polyureas, polyphosphazenes, polysilazanes, polypropylenes, polyethylenes, polyethylene terephthalates, polyvinylidene fluorides, polysiloxanes, or a combination thereof.

8. The fragrance composition of claim 1, where the synthetically derived organic polymer is a thermoset and comprises epoxy polymers, unsaturated polyester polymers, polyimide polymers, bismaleimide polymers, bismaleimide triazine polymers, cyanate ester polymers, vinyl polymers, benzoxazine polymers, benzocyclobutene polymers, acrylics, alkyds, phenol-formaldehyde polymers, novolacs, resoles, melamine-formaldehyde polymers, urea-formaldehyde polymers, hydroxymethylfurans, isocyanates, diallyl phthalate, triallyl cyanurate, triallyl isocyanurate, unsaturated polyesterimides, resorcinol formaldehyde, phenol formaldehyde, melamine formaldehyde, cresol formaldehyde, phenol furfuryl alcohol or a combination thereof.

9. The fragrance composition of claim 1, where the synthetically derived organic polymer comprises an oligomer, a homopolymer, a copolymer, a block copolymer, an alternating block copolymer, a random polymer, a random copolymer, a random block copolymer, a graft copolymer, a star block copolymer, a dendrimer, a polyelectrolyte, a polyampholyte, an ionomer, or a combination thereof.

10. The fragrance composition of claim 1, where the active fragrance compound comprises lactic acid, 2-hydroxydecanoic acid, 2-hydroxyoctanoic acid and glycolic acid, beta-hydroxy acids such as beta-hydroxy salicylic acid, avobenzone, benzoate-4-methylbenzylidene, cinoxate, dioxybenzone, homosalate, menthyl anthranilate, mexoryl SX, drometrizole trisiloxane, octocrylene, octyl methoxycinnamate, ethylhexyl salicylate, oxybenzone, padimate O, p-aminobenzoic acid (PABA), phenylbenzimidazole sulfonic acid, sulisobenzone, titanium trolamine salicylate, salicylic acid, a retinoic acid, benzoyl peroxide, hydroquinone, arbutun, a plant extract containing arbuten, kojic acid, azelaic acid, glycyrrhetic acid, levulinic acid, 2-cyano-3,3-diphenylacrylic acid, sodium benzotriazolyl butylphenol sulfonate, ethyl-2-cyan-3,3-diphenylacrylate, 2-t-butyl-6-(5-chloro-2H-benzotriazol-2-yl)-p-cresol, 2-(2-H-benzotriazol-2-yl)-4-methylphenol, benzophenone-12, bornelone, 2-benzotriazolyl-4-tert-octylphenol, dihydroxyacetone, erythrulose, dihydroxyacetone-ortho-ethyl acetate, canthaxanthin, afamelanotide, minoxidil, finasteride, dutasteride, ketoconazole, zinc pyrithione, selenium sulfide, clotrimazole, tea tree oil, piroctone olamine, an amphetamine, an antihistamine, methylphenidate, oxymetazoline, tetrahydrolzoline hydrochloride, psilocybin calcium, thioglycolate, sodium thioglycolate, thioglycolic acid, ammonium thioglycolate, butyl thioglycolate, ethanolamine thioglycolate, glyceryl thioglycolate, isooctyl thioglycolate, isopropyl thioglycolate, magnesium thioglycolate, methyl thioglycolate, potassium thioglycolate, aluminum zirconium tetrachlorohydrex gly, aluminum chlorohydrate, aluminum chloride, resorcinol, 1-napthol, p-aminophenol, p-phenylenediamine, salts of p-phenylenediamine, 4-amino-2-hydroxytoluene, phenoxyethanol, N,N-diethyl-m-toluamide, p-menthane-3,8-diol, nepetalactone, citronella oil, permethrin, neem oil, bog myrtle extract, or a combination thereof.

11. The fragrance composition of claim 1, where the active fragrance compound comprises cottonseed oil, lavender essential oil, vanilla oil, cinnamon oil, citronella oil, goji berry fragrance oil, orange oil, mandarin orange oil, apple seed oil, Caribbean teakwood oil, cedar oil, sandalwood oil, juniper oil, nutmeg oil, star anise oil, patchouli oil, rose oil, clove oil, saffron oil, lavender oil, rosemary oil, clary sage oil, lemon oil, peppermint oil, sweet basil oil, bergamot oil, camellia oil (or tea seed oil), blue chamomile oil, catnip oil, bay leaf oil, clementine oil, coffee essential oil, coconut oil, oregano oil, ylang-ylang oil, neroli essential oil, bergamot essential oil, rose petal oil, jasmine essential oil, vetiver essential oil, citrus essential oil, olibanum essential oil, petitgrain oil, sweet orange oil, myrrh essential oil, coriander seed oil, frankincense oil, white musk, ambergris, civet musk, hyraceum, beeswax, or a combination thereof.

12. A method of manufacturing a fragrance composition comprising:
blending together an organic aerogel and a non-edible active fragrance compound to form the fragrance composition; where the organic aerogel comprises biopolymer or a synthetically derived organic polymer; where the organic aerogel does not contain any inorganic aerogels and wherein the organic aerogels are not blended with or dispersed in hydrogels, inorganic aerogels or with non-aerogel polymers.

13. A method comprising:
exposing the fragrance composition of claim 1 to an atmosphere to discharge the active fragrance compound to the ambient atmosphere.

14. A fragrance emitting device comprising:
a first container that is operative to hold the fragrance composition of claim 1; where the first container comprises porous walls that are operative to discharge the active fragrance compound to a surrounding atmosphere; or alternatively, where the first container comprises non-porous walls and where the non-porous walls contain one or more ports through which the active fragrance compound may be sampled.

15. The fragrance emitting device of claim 14, further comprising a second container; where the second container is in operative communication with and lies upstream of the first container; where the second container stores a fragrance composition that is discharged to the first container.

16. The fragrance emitting device of claim 15, wherein the fragrance composition is discharged from the second container to the first container using gravity or mechanical force.

17. The fragrance emitting device of claim 15, further comprising a third container that lies downstream of the first container; where the third container is operative to receive a spent fragrance composition from the first container.

18. The fragrance emitting device of claim 16, further comprising a passage disposed between the first container and the third container, where the passage has a cross-sectional area that permits the spent fragrance composition to be transported through when an organic aerogel particle has attained a desired size.

19. The fragrance emitting device of claim 14, further comprising a chamber that is in operative communication with the first container; where the chamber comprises a heater and/or a first source of forced air; where the heater is operative to heat air supplied to the first container and where the first source of forced air is used to regulate air supply to the first container.

20. The fragrance emitting device of claim 14, further comprising a mobile chamber that comprises the fragrance composition; where the mobile chamber can be moved from a position where it is not exposed to an ambient atmosphere to another position where it is exposed to ambient atmosphere and discharges the active fragrance compound to the ambient atmosphere.

21. The fragrance emitting device of claim 14, wherein the fragrance emitting device emits the active fragrance compound from the fragrance composition to the atmosphere such that a user perceives that the surrounding atmosphere contains a constant level of the active fragrance compound with the passage of time.

22. The fragrance emitting device of claim 14, wherein the fragrance emitting device emits an active fragrance compound whose composition varies with time.

23. The fragrance emitting device of claim 14, wherein the fragrance emitting device emits a first active fragrance compound for a first period of time and a second active fragrance compound for a second period of time; where the first active fragrance compound is different from the second active fragrance compound and where the second period of time succeeds the first period of time.

24. The fragrance emitting device of claim 20, wherein the active fragrance compound is subjected to a circumferentially directed air flow; where the circumferentially directed air flow can contact the fragrance composition a plurality of times during a travel of the air flow in the fragrance emitting device.

25. The fragrance emitting device of claim 20, where the mobile chamber comprising the fragrance composition can be moved from a position where it is exposed to an ambient atmosphere and discharges the active fragrance compound to the ambient atmosphere to another position where it is not exposed to ambient atmosphere and does not discharge the active fragrance compound to the ambient atmosphere.

26. The fragrance emitting device of claim 14, wherein the fragrance emitting device emits the active fragrance compound from the fragrance composition to the atmosphere such that the amount of fragrance emitted is controlled by varying exposure to air and heat to create a variable or constant user perception.

27. The fragrance emitting device of claim 14, where the active fragrance compound is inhaled directly by a user through the one or more ports.

28. A fragrance emitting device comprising:
a first container comprising a porous wall or a non-porous wall that is operative to contain an active fragrance compound that can be discharged to a surrounding atmosphere; and
a mobile chamber that comprises the active fragrance compound; where the mobile chamber can be moved from a position where it is not exposed to an ambient atmosphere to another position where it is exposed to ambient atmosphere and discharges the active fragrance compound to the ambient atmosphere.

29. The fragrance emitting device of claim 28, wherein the active fragrance compound is subjected to a circumferentially directed air flow; where the circumferentially directed air flow can contact the fragrance composition a plurality of times during a travel of the air flow in the fragrance emitting device.

30. The fragrance emitting device of claim 28, where the mobile chamber can be moved from a position where it is exposed to an ambient atmosphere and discharges the active fragrance compound to the ambient atmosphere to another position where it is not exposed to ambient atmosphere and does not discharge the active fragrance compound to the ambient atmosphere.

31. The fragrance emitting device of one of claim 14, 19 or 20, further comprising a second source of forced air that is combined with the air exiting the fragrance compound chamber; and wherein the second source of forced air is directed in a different direction from a first source of forced air.
